(19) United States
(12) Patent Application Publication
(10) Pub. No.: US 2008/0135419 A1
(43) Pub. Date: Jun. 12, 2008

(54) Hdl Cholesterol Sensor

(76) Inventors: Patricia Mary Elizabeth Roblin, Yarnton (GB); John Morton Broughall, Yarnton (GB); Luet Lok Wong, Yarnton (GB)

Correspondence Address: QUARLES & BRADY LLP, 411 E. WISCONSIN AVENUE, SUITE 2040, MILWAUKEE, WI, 53202-4497, US

(21) Appl. No.: 11/793,285

(22) PCT Filed: Dec. 12, 2005
(86) PCT No.: PCT/GB05/04952

§ 371 Date: Aug. 2, 2007

(30) Foreign Application Priority Data
Dec. 22, 2004 (GB) 0428130.9

(51) Int. Cl. G01N 27/26; G01N 33/92; C12Q 1/60

(52) U.S. Cl. 205/777.5; 436/71; 435/11; 204/403.14

(57) ABSTRACT

A method for the determination of the amount of cholesterol in high density lipoproteins in a high density lipoprotein containing sample, the method comprising reacting the sample with a PEG-ylated protein to selectively complex non-HDL lipoproteins in the sample with the PEG-ylated protein, or with a PEG-ylated enzyme capable of selective reaction with high density lipoproteins, and subsequently measuring the amount of cholesterol in the high density lipoproteins, for example using an electrochemical technique.

### FIELD OF THE INVENTION

The present invention relates to a method for determining the amount of cholesterol bound to high density lipoproteins (HDL-cholesterol) in a high density lipoprotein-(HDL-) containing sample. The invention also relates to a composition and a kit for use in such a method.

### BACKGROUND OF THE INVENTION

Many epidemiological investigations have demonstrated the strong and independent inverse association of high density lipoprotein (HDL), measured in terms of either its cholesterol or apo AI content, to risk of coronary artery disease (CAD). It is said that the risk of CAD increases 2-3% for every 10 mg/L decrease in HDL-cholesterol. Thus, higher HDL-cholesterol concentrations are considered protective. The measurement of HDL-cholesterol in characterizing risk for CAD and managing treatment of dyslipidemia has therefore become increasingly common in clinical laboratories.

Initial laboratory methods for HDL-cholesterol measurement, adapted from research techniques, required a manual separation step with precipitation reagents, followed by analysis of the cholesterol content, most often by an automated chemistry analyzer. Typical separation steps involved the reaction of a precipitation reagent with low density lipoproteins (LDL), very low density lipoproteins (VLDL) and chylomicrons (CM) in order to form an aggregate of these components. The aggregate was then removed from the reaction vessel, for example by centrifugation, leaving an HDL-containing sample ready for analysis. Separation of the precipitate was essential in order that the precipitate did not interfere with the UV/Vis or colorimetric analysis techniques used.

A number of so-called “homogeneous” methods have also been developed. In these methods, separation of the precipitated lipoproteins can be avoided by, for example, adding a clearing reagent to dissolve the precipitate after reaction with HDL-cholesterol is completed. In this way, the LDL, VLDL and CM are blocked ensuring selective reaction with HDL-cholesterol, but are cleared prior to carrying out the UV/Vis analysis. Alternatively, specific reaction conditions such as high dilution, or specific precipitation reagents, are used to ensure minimum interference with the analysis technique.

Typically, such methods involve the addition of two or more reagents to a sample, with incubation periods after addition of the reagents, followed by a measurement step, e.g. by colorimetric development or by UV/Vis analysis. However, whilst these methods may now be automated, they still require a relatively complex analysis procedure to be completed, with several different reagents needing to be added at different times. Therefore, analysis requires specialist equipment and can typically only be carried out in a clinical laboratory by a skilled technician. Further, the potential need for relatively long incubation periods to allow the reagents to react leads to a time delay in obtaining measurements.

A further problem with some known HDL-cholesterol detection techniques is that they cannot be carried out on whole blood. The hematocrit tends to interfere with the UV or visible analysis techniques and a reliable result cannot be obtained. Similar difficulties occur using colorimetric analysis techniques. Haemolysis is a further problem which is encountered when spectroscopic analysis is used. Measurement is therefore generally carried out on serum or plasma samples. Furthermore, the use of neat serum or plasma samples is not possible since the extinction coefficients of such systems are far too high for an adsorption measurement to be taken. High dilution of samples is therefore required. Measurement of the HDL-cholesterol content of a whole blood sample therefore cannot be completed without either first carrying out one or more pre-treatment steps or using highly dilute reagents. This further increases the requirement for the measurement to be carried out by a skilled technician.

There is therefore a need for simple, effective and rapid methods for analysing the HDL-cholesterol content of body fluids such as blood or plasma. Any such method should be capable of effectively distinguishing between cholesterol bound to HDL, and cholesterol bound to low density lipoproteins (LDL), very low density lipoproteins (VLDL) and chylomicrons (CM), thus providing a method which is selective for HDL-cholesterol. Further, preferred methods will not employ specialist equipment, or require trained technicians to carry out. A technique which avoids manual separation steps and lessens the requirements for pre-treatment, e.g. dilution, of a sample is also needed.

### SUMMARY OF THE INVENTION

The present invention provides a method for the determination of the amount of cholesterol in high density lipoproteins in a high density lipoprotein containing sample, said method comprising reacting the sample with a PEG-ylated protein, which typically selectively complexes non-HDL lipoproteins in the sample with said PEG-ylated protein, and subsequently measuring the amount of cholesterol in the high density lipoproteins.

PEG-ylated proteins have surprisingly been found to be particularly effective in complexing with non-HDL lipoproteins (typically LDL and VLDL or LDL, VLDL and CM) in a sample, but do not complex with HDL. Thus, it is thought that the addition of a PEG-ylated protein to the sample blocks the non-HDL lipoproteins from participating in any cholesterol assay which is carried out, and thereby enables the skilled person to selectively test for HDL-cholesterol. Typically, the HDL-cholesterol content of the sample is then measured by reacting with a cholesterol ester hydrolysing reagent as well as either cholesterol oxidase or cholesterol dehydrogenase, and measuring the amount of cholesterol which has reacted with the cholesterol oxidase or cholesterol dehydrogenase, for example by an electrochemical technique.

In a preferred embodiment, the protein is an enzyme-stabilising protein such as albumin, in particular bovine serum albumin (BSA). Thus, preferred PEG-ylated proteins include PEG-ylated albumin, most preferably PEG-ylated BSA. This embodiment is particularly advantageous as the PEG-ylated protein may have the dual effect of stabilising any enzyme reagents which are used to carry out the cholesterol assay, as well as complexing with the non-HDL lipoproteins.

In an alternative embodiment, the invention provides an electrochemical technique for the measurement of HDL-cholesterol in a sample, which is not limited to the use of any specific precipitating agent and does not require the use of particular reaction conditions for precipitation of the LDL, VLDL and CM. In this embodiment, one of the enzymes involved in the reaction is PEG-ylated, providing selective reaction with HDL over other lipoproteins. A PEG-ylated protein and/or a complexing reagent capable of forming a complex with non-HDL lipoproteins may also be used, if desired.

Accordingly, the present invention also provides an electrochemical method for the determination of the amount of cholesterol in high density lipoproteins in a high density lipoprotein containing sample, said method comprising reacting the sample with
(b) a cholesterol ester hydrolysing reagent;
(c) cholesterol oxidase or cholesterol dehydrogenase;
(d) a coenzyme;
(e) a redox agent capable of being oxidised or reduced to form a product; and optionally
(f) a surfactant, and/or
(h) a complexing reagent capable of forming a complex with low density and very low density lipoproteins; and/or
(i) a PEG-ylated protein;
   and electrochemically detecting the amount of product formed, wherein the cholesterol ester hydrolysing reagent and/or cholesterol oxidase or cholesterol dehydrogenase is modified with polyethylene glycol.

The use of electrochemical analysis provides a rapid and very simple test which gives reliable measurements of HDL-cholesterol content. The test can be carried out in a single step and does not require the addition of separate reagents. Furthermore, specialist equipment is not required.

In one embodiment of the invention, the electrochemical test can be carried out on a portable hand-held device and is therefore particularly easy to use in a medical environment, for example in a doctor's surgery, a hospital room or ward, or by the patient themselves at home. No skilled technician is required. Furthermore, the technique of the present invention provides results in a short period of time, in some cases in under five minutes.

The present invention can also be carried out on a neat sample, for example a neat plasma or serum sample. Furthermore, the method of the present invention may also comprise a step of filtering the sample to remove red cells from whole blood, thus enabling the method to be directly carried out on neat whole blood samples. Thus, the method of the invention requires no pre-treatment steps and can easily be carried out by an unskilled user.

The present invention also provides a reagent mixture for use in carrying out an assay of the invention, the reagent mixture comprising
(a) a PEG-ylated protein, which is e.g. capable of selectively complexing with non-HDL lipoproteins;
(b) a cholesterol ester hydrolysing reagent;
(c) cholesterol oxidase or cholesterol dehydrogenase; and optionally
(f) a surfactant.

Also provided by the present invention is a reagent mixture for use in an electrochemical method for the determination of the amount of cholesterol in high density lipoproteins in a high density lipoprotein containing sample, the reagent mixture comprising
(b) a cholesterol ester hydrolysing reagent;
(c) cholesterol oxidase or cholesterol dehydrogenase;
(d) a coenzyme;
(e) a redox agent capable of being oxidised or reduced to form a product; and optionally
(h) a complexing reagent capable of forming a complex with low density and very low density lipoproteins; and/or
(i) a PEG-ylated protein;
   wherein the cholesterol ester hydrolysing reagent and/or the cholesterol oxidase or cholesterol dehydrogenase is modified with polyethylene glycol.

The present invention also provides a kit for the determination of the amount of cholesterol in high density lipoproteins in a high density lipoprotein containing sample, the kit comprising (a) a PEG-ylated protein capable of selectively complexing with non-HDL lipoproteins, (b) a cholesterol ester hydrolysing reagent, (c) cholesterol oxidase or cholesterol dehydrogenase, optionally (f) a surfactant, and means for measuring the amount of cholesterol which reacts with the cholesterol oxidase or cholesterol dehydrogenase.

Also provided is a kit comprising
an electrochemical cell having a working electrode, a reference or pseudo reference electrode and optionally a separate counter electrode;
the reagents as described above, (e.g. (a) a PEG-ylated protein capable of selectively complexing with non-HDL lipoprotens, (b) a cholesterol ester hydrolysing reagent, (c) cholesterol oxidase or cholesterol dehydrogenase, (d) a coenzyme, (e) a redox agent capable of being oxidised or reduced to form a product; and optionally (f) a surfactant and/or (g) a reductase);
a power supply for applying a potential across the cell; and
a measuring instrument for measuring the resulting electrochemical response, e.g. the current across the cell.

Thus, the present invention provides a kit for determining the HDL-cholesterol content of a sample by electrochemical means. This kit is particularly simple to use. A user merely needs to add the sample to be tested, apply a potential across the cell and measure the generated current. There is therefore no need for additional steps involving the addition of specific quantities of reagents. Furthermore, the device can contain a predetermined amount of each of the required reagents, avoiding the need for the user to measure out particular quantities of material.

In a further preferred embodiment, the electrochemical cell is in the form of a receptacle or partial receptacle, the working electrode is in a wall of the receptacle or partial receptacle and the reagents (e.g. reagents (a) to (e) and optionally (f) and/or (g)) are at least partly contained within the receptacle or partial receptacle. In an alternative preferred embodiment, the device comprises a strip having at least one receptacle or partial receptacle formed therein, the receptacle or partial receptacle having a first open part in a first surface of the strip to enable a sample to enter the receptacle or partial receptacle,
wherein the working electrode of the electrochemical cell is in a wall of the receptacle or partial receptacle, and
wherein the reference or pseudo reference electrode of the electrochemical cell comprises a reference or pseudo reference electrode layer formed on at least a part of the first surface of the strip, and
wherein the reagents (e.g. reagents (a) to (e) and optionally (f) and/or (g)) are at least partly contained within the receptacle or partial receptacle.

The present invention also provides a method of operating the kit of the invention, the method comprising
(i) contacting (1) the reagents as described above (e.g. the PEG-ylated protein, cholesterol ester hydrolysing reagent, cholesterol oxidase or cholesterol dehydrogenase, coenzyme, redox agent and optionally surfactant and/or reductase), and (2) a high density lipoprotein containing sample, with each other and with the electrodes;
(ii) applying a potential across the electrochemical cell; and
(iii) electrochemically detecting the amount of product formed by measuring the resulting electrochemical response, e.g. the current across the cell.

The present invention further provides the use of a PEG-ylated protein as a selective non-HDL lipoprotein complexing agent, typically a selective LDL, VLDL and CM complexing agent, in a method for the determination of the amount of cholesterol in high density lipoproteins in a high density lipoprotein containing sample.

### BRIEF DESCRIPTION OF THE FIGURES

FIG. 1 depicts a device according to one embodiment of the invention;

FIG. 2 depicts an alternative device according to the invention.

### DETAILED DESCRIPTION OF THE INVENTION

The present invention provides a method of selectively determining the HDL-cholesterol content of a sample, wherein the sample may contain other lipoproteins which bind to cholesterol, as well as HDL. In one embodiment, the method involves reacting the sample with a PEG-ylated protein capable of selectively complexing with non-HDL lipoproteins. Typically the PEG-ylated protein is capable of selectively complexing with LDL, VLDL and CM. The HDL in the sample does not complex with such PEG-ylated proteins. Thus, it is thought that the use of a PEG-ylated protein blocks the non-HDL lipoproteins from taking part in any cholesterol assay, whilst leaving HDL available for reaction. The PEG-ylated protein may form a 1:1 complex with the non-HDL lipoproteins, or it may form larger aggregates, for example precipitates which may be insoluble in the sample.

Examples of proteins for use in the PEG-ylated protein reagent include polyaminoacids (e.g. polylysine), gelatine, lysozyme, lactalbumin and serum albumin (e.g. bovine serum albumin). Preferred proteins for use in the PEG-ylated protein reagent include proteins having enzyme stabilising activity. For example, albumins, in particular bovine serum albumin (BSA), are preferred proteins due to their enzyme stabilising properties. The benefit of using such a protein is that any enzymes used in the cholesterol assay may be stabilised by the PEG-ylated protein, without the need to add further stabilisers (although further stabilisers can be used if desired). When the assay is carried out on a blood sample, albumin proteins have the further advantage that they are present in high quantities in blood and are therefore typically inert to any cholesterol assay appropriate for testing blood. Proteins other than albumins, which are also inert to the cholesterol assay to be used are also envisaged.

Typically, the concentration of the PEG-ylated protein when mixed with the sample to be tested is approximately 1 to 10%, for example about 5% w/v.

PEG-ylation of the protein is typically carried out in accordance with routine procedures in the art. Typically, the PEG is activated prior to use to enable addition to the protein. For example, PEG may first be activated to form PEG propionic acid which is then esterified with a succinimidyl group. Such activated products are available, for example, from Nektar Therapeutics USA. PEG is then reacted with the protein, typically in a ratio of from 1:1 to 1:8, preferably 1:2. Typically, PEG with a molecular weight of from 2 to 30 k, for example 4 to 20 k or 5 to 10 k is used.

A surfactant may be used in the method of the invention in order to break down the HDL and to ensure that all of the cholesterol or cholesterol esters bound to HDL are available for reaction. Examples of suitable surfactants include polyoxyethylene derivatives such as polyoxyethylene alkylene tribenzyl phenyl ether and polyoxyethylene alkylene phenyl ether. A preferred surfactant is B66 (Kao Corporation). Surfactants are typically used in an amount of up to 500 mg per ml of sample to be tested, preferably up to 200 mg/ml, for example about 50 mg/ml.

The measurement of the HDL-cholesterol content of the sample may be carried out by any suitable technique for measuring cholesterol. A preferred technique involves the reaction of the sample with a cholesterol ester hydrolysing reagent and cholesterol oxidase or cholesterol dehydrogenase. The cholesterol contained in HDL lipoproteins may be in the form of free cholesterol or cholesterol esters. The cholesterol ester hydrolysing reagent is therefore used to break down any cholesterol esters into free cholesterol. The free cholesterol is then reacted with the cholesterol oxidase or cholesterol dehydrogenase and the amount of cholesterol which has undergone such reaction is measured.

The cholesterol ester hydrolysing reagent may be any reagent capable of hydrolysing cholesterol esters to cholesterol. The reagent should be one which does not interfere with the reaction of cholesterol with cholesterol oxidase or cholesterol dehydrogenase and any subsequent steps in the assay. Preferred cholesterol ester hydrolysing reagents are enzymes, for example cholesterol esterase and lipases. A suitable lipase is, for example, a lipase from a pseudomonas or chromobacterium viscosum species. Commercially available enzymes, optionally containing additives such as stabilisers or preservatives may be used, e.g. those available from Toyobo or Amano. The cholesterol ester hydrolysing reagent may be used in an amount of from 0.1 to 20 mg per 100 μl of sample, preferably from 0.5 to 15 mg per 100 μl. Any commercially available forms of cholesterol oxidase and cholesterol dehydrogenase may be employed. For instance, the cholesterol dehydrogenase is, for example, from the Nocardia species. The cholesterol oxidase or cholesterol dehydrogenase may be used in an amount of from 0.01 mg to 100 mg per 100 μL of reagent mixture. In one embodiment, the cholesterol oxidase or dehydrogenase is used in an amount of from 0.1 to 20 mg per 100 μl of sample, preferably from 0.5 to 10 mg per 100 μl.

Each of the enzymes may contain additives such as stabilisers or preservatives. The need for stabilisers may be reduced or avoided if PEG-ylated BSA is used as the PEG-ylated protein. Further, each of the enzymes may be chemically modified. For example, they may be PEG-ylated enzymes as described below.

The PEG-ylated protein may be added to the sample prior to addition of the other reagents or simultaneously with the addition of the other reagents. In a preferred embodiment, the cholesterol ester hydrolysing reagent, cholesterol oxidase or dehydrogenase and PEG-ylated protein are present in a single reagent mixture which is combined with the sample in a single step.

Measurements in accordance with the present invention can be carried out on any suitable sample containing HDL-cholesterol. Measurements are typically carried out on whole blood or blood components, for example serum or plasma. Preferred samples for use in the method of the present invention are serum and plasma. Where measurements are to be carried out on whole blood, the method may include the additional step of filtering the blood to remove red blood cells.

In a preferred embodiment of the invention, an electrochemical technique is used to measure the HDL-cholesterol content. In this embodiment, the sample is typically reacted with the PEG-ylated protein, a cholesterol ester hydrolysing reagent, cholesterol oxidase or cholesterol dehydrogenase, a coenzyme capable of interacting with cholesterol oxidase or cholesterol dehydrogenase, and a redox agent which is capable of being oxidised or reduced to form a product which can be electrochemically detected at an electrode. The mixture of sample and reagents is contacted with a working electrode of an electrochemical cell so that redox reactions occurring can be detected. A potential is applied across the cell and the resulting electrochemical response, typically the current, is measured.

In this preferred embodiment, the amount of HDL-cholesterol is measured in accordance with the following assay:

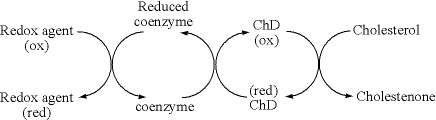

where ChD is cholesterol dehydrogenase. Cholesterol dehydrogenase could be replaced with cholesterol oxidase in this assay if desired. The amount of reduced redox agent produced by the assay is detected electrochemically. Additional reagents may also be included in this assay if appropriate.

Typically, the sample contacts all of the reagents in a single step. Therefore, a reagent mixture is provided which contains all of the required reagents and which can easily be contacted with the sample in order to carry out the assay. The reagent mixture of the invention typically comprises from 1 to 10% w/v (1 to 10 mg per 100 μl) preferably from 2 to 8% (2 to 8 mg per 100μ) of PEG-ylated protein, from 0.1 to 20 mg per 100 μl, preferably from 0.5 to 10 mg per 100 μl of cholesterol ester hydrolysing reagent and from 0.1 to 20 mg per 100 μl, preferably from 0.5 to 10 mg per 100 μl of cholesterol dehydrogenase.

Typically the coenzyme is NAD⁺ or an analogue thereof. An analogue of NAD⁺ is a compound having structural characteristics in common with NAD⁺ and which also acts as a coenzyme for cholesterol dehydrogenase. Examples of NAD⁺ analogues include APAD (Acetyl pyridine adenine dinucleotide); TNAD (Thio-NAD); AHD (acetyl pyridine hypoxanthine dinucleotide); NaAD (nicotinic acid adenine dinucleotide); NHD(nicotinamide hypoxanthine dinucleotide); and NGD (nicotinamide guanine dinucleotide). The coenzyme is typically present in the reagent mixture in an amount of from 1 to 20 mM, for example from 3 to 15 mM, preferably from 5 to 10 mM.

Typically, the redox agent should be one which can be reduced in accordance with the assay shown above. In this case, the redox agent should be one which is capable of accepting electrons from a coenzyme (or from a reductase as described below) and transferring the electrons to an electrode. The redox agent may be a molecule or an ionic complex. It may be a naturally occurring electron acceptor such as a protein or may be a synthetic molecule. The redox agent will typically have at least two oxidation states.

Preferably, the redox agent is an inorganic complex. The agent may comprise a metallic ion and will preferably have at least two valencies. In particular, the agent may comprise a transition metal ion and preferred transition metal ions include those of cobalt, copper, iron, chromium, manganese, nickel, or ruthenium. The redox agent may be charged, for example it may be cationic or alternatively anionic. An example of a suitable cationic agent is a ruthenium complex such as Ru(NH₃)₆³⁺, an example of a suitable anionic agent is a ferricyanide complex such as Fe (CN)₆³⁻.

Examples of complexes which may be used include Cu(EDTA)²⁻, Fe(CN)₆³⁻, Fe(CN)₅(O₂CR)³⁻, Fe(CN)₄(oxalate)³⁻, Ru(NH₃)₆³⁺ and chelating amine ligand derivatives thereof (such as ethylenediamine), Ru(NH₃)₅(py)³⁺, ferrocenium and derivatives thereof with one or more of groups such as —NH₂, —NHR, —NHC(O)R, and —CO₂H substituted into one or both of the two cyclopentadienyl rings. Preferably the inorganic complex is Fe(CN)₆³⁻, Ru(NH₃)₆³⁺, or ferrocenium monocarboxylic acid (FMCA).

The redox agent is typically present in the reagent mixture in an amount of from 10 to 200 mM, for example from 30 to 150 mM, preferably from 50 to 100 mM.

In one embodiment of the invention, the redox agent also acts as a complexing agent. Ru³⁺compounds, for example Ru(NH₃)₆³⁺, are particularly preferred redox agents for use in this embodiment. Thus, the PEG-ylated protein and redox agent may be used in combination to selectively complex non-HDL lipoproteins. The redox agent, in particular Ru³⁺ compounds, may thus be employed as a complexing agent in any assay of the invention including non-electrochemical assays.

In a preferred embodiment, the reagent mixture used in the electrochemical assay additionally comprises a reductase. The reductase typically transfers two electrons from the reduced NAD and transfers two electrons to the redox agent. The use of a reductase therefore provides swift electron transfer.

Examples of reductases which can be used include diaphorase and cytochrome P450 reductases, in particular, the putidaredoxin reductase of the cytochrome P450_{cam} enzyme system from Pseudomonas putida, the flavin (FAD/FMN) domain of the P450_{BM-3} enzyme from Bacillus megaterium, spinach ferrodoxin reductase, rubredoxin reductase, adrenodoxin reductase, nitrate reductase, cytochrome b₅ reductase, corn nitrate reductase, terpredoxin reductase and yeast, rat, rabbit and human NADPH cytochrome P450 reductases. Where a nitrate reductase is employed preferably corn nitrate reductase is used. Preferred reductases for use in the present invention include diaphorase and putidaredoxin reductases.

The reductase may be a recombinant protein or a naturally occurring protein which has been purified or isolated. The reductase may have been mutated to improve its performance such as to optimise the speed at which it carries out the electron transfer or its substrate specificity.

The reductase is typically present in the reagent mixture in an amount of from 0.5 to 100 mg/ml, for example from 1 to 50 mg/ml, 1 to 30 mg/ml or from 5 to 20 mg/ml.

In a preferred embodiment of the invention, the general scheme of the electrochemical assay is as follows:

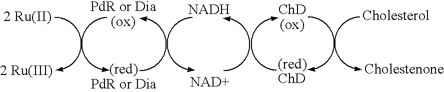

### Where

PdR—is putidaredoxin reductase
Dia—is diaphorase
ChD—is cholesterol dehydrogenase.

The reagent mixture optionally contains one or more additional components, for example surfactants as described above and/or excipients and/or buffers and/or stabilisers. Excipients are preferably included in the reagent mixture in order to stabilize the mixture and optionally, where the reagent mixture is dried onto the device of the invention, to provide porosity in the dried mixture. Examples of suitable excipients include sugars such as mannitol, inositol and lactose, and PEG. Glycine can also be used as an excipient. Buffers may also be included to provide the required pH for optimal enzyme activity. For example, a Tris buffer (pH9) may be used. Stabilisers may be added to enhance, for example, enzyme stability. Examples of suitable stabilisers are amino acids, e.g. glycine, and ectoine.

In a preferred embodiment, the reagent mixture for the electrochemical assay of the invention comprises a PEG-ylated protein selected from polyaminoacids, gelatine, lysozyme, lactalbumin and serum albumin; cholesterol esterase or a lipase; cholesterol dehydrogenase; NAD⁺or an analogue thereof; a reductase; and a redox agent. In a more preferred embodiment, the reagent mixture comprises PEG-ylated BSA, cholesterol esterase or a lipase, cholesterol dehydrogenase, NAD⁺or an analogue thereof, diaphorase or putidaredoxin reductase and Ru(NH₃)₆³⁺.

An alternative reagent mixture for use in the electrochemical assay of the invention is also provided. In this embodiment, the cholesterol ester hydrolysing reagent and/or the cholesterol oxidase or cholesterol dehydrogenase is PEG-ylated, providing the necessary selectivity for HDL over other lipoproteins. The inclusion of a further PEG-ylated protein is therefore an optional feature of this embodiment. In addition to the cholesterol ester hydrolysing reagent and cholesterol oxidase or cholesterol dehydrogenase, the sample is also reacted with a coenzyme capable of interacting with cholesterol oxidase or cholesterol dehydrogenase, a redox agent which is capable of being oxidised or reduced to form a product which can be electrochemically detected at an electrode, and optionally a surfactant and/or a reductase. These additional reagents are typically as described above for the first embodiment of the invention.

The mixture of sample and reagents is contacted with a working electrode of an electrochemical cell so that redox reactions occurring can be detected. A potential is applied across the cell and the resulting current is measured. The amount of HDL-cholesterol is typically measured in accordance with the redox assays described above. Typically, all of the reagents contact the sample in a single step. A single reagent mixture is therefore provided comprising all of the required reagents.

The cholesterol ester hydrolysing reagent is typically an enzyme, for example cholesterol esterase or a lipase as described above. The enzyme used as the cholesterol ester hydrolysing reagent and/or the cholesterol oxidase or cholesterol dehydrogenase is modified with PEG, e.g. PEG having a molecular weight of from 3000 to 6000. In a preferred embodiment, the cholesterol ester hydrolysing reagent is a PEG-ylated enzyme and cholesterol dehydrogenase which is either unmodified or modified is used. In a further preferred embodiment, the cholesterol ester hydrolysing reagent is a PEG-ylated cholesterol esterase or lipase and a modified or unmodified cholesterol dehydrogenase is used.

The sample may also be reacted with a PEG-ylated protein as described above and/or with a complexing reagent capable of forming a complex with non-HDL lipoproteins, typically LDL and VLDL. The complexing reagent may also form a complex with chylomicrons (CM). Once in complexed form, the LDL, VLDL and CM are unavailable for reaction with enzymes and therefore do not interfere with the assay described above. In this way, the assay has further selectivity for HDL-cholesterol. Due to the use of PEG-ylated reagents, however, such complexing agent is not essential.

The complexing reagent may form a 1:1 complex with the LDL, VLDL or CM, or it may form larger aggregates, for example precipitates which may be insoluble in the sample. Such insoluble precipitates do not interfere with the electrochemical detection step. Examples of complexing agents include polyanions, combinations of polyanions with divalent metal salts, and antibodies capable of binding to apob containing lipoproteins. The polyanions may be selected from phosphotungstic acid and salts thereof, dextran sulphuric acid and salts thereof, polyethylene glycol and heparin and salts thereof, and are typically present in the reagent mixture in an amount of up to 200 mM, e.g. from 10 to 200 mM, for example from 30 to 150 mM, preferably from 50 to 100 mM. Phosphotungstic acid and its salts are preferred polyanions. The divalent metal salts include the salts of Group IIA metals, e.g. Mg and Ca, and Mn, and are typically present in the reagent mixture in an amount of from 10 to 200 mM, for example from 30 to 150 mM, preferably from 50 to 100 mM. Mg is a preferred metal. The anion is typically a halide such as chloride, or a sulfate. MgCl₂ and MgSO₄ are preferred divalent metal salts.

In a preferred aspect of this embodiment, the reagent mixture comprises a PEG-modified cholesterol esterase or a PEG-modified lipase, cholesterol dehydrogenase, NAD+ or an analogue thereof, a reductase, a redox agent and optionally a complexing reagent or PEG-ylated protein. In a more preferred embodiment, the reagent mixture comprises a PEG-modified cholesterol esterase or a PEG-modified lipase, cholesterol dehydrogenase, NAD+ or an analogue thereof, diaphorase or putidaredoxin reductase, Ru(NH₃)₆³⁺ and optionally either phosphotungstic acid and a divalent metal salt, e.g. MgCl₂, or PEG-ylated BSA.

The reagent mixture of the invention is typically provided in solid form, for example in dried form, or as gel. Alternatively it may be in the form of a solution or suspension. Whilst the amounts of each of the components present in the reaction mixture are expressed above in terms of molarity or w/v, the skilled person would be able to adapt these amounts to suitable units for a dried mixture or gel, so that the relative amounts of each component present remains the same.

Where an electrochemical measurement is carried out on whole blood, the measurement obtained may depend on the hematocrit. The measurement should therefore ideally be adjusted to at least partially account for this factor. Alternatively, the red blood cells can be removed by filtering the sample prior to carrying out the assay.

The present invention also provides a kit for selectively determining the HDL-cholesterol content of an HDL-containing sample. The kit includes the required reagents, e.g. the PEG-ylated protein, cholesterol ester hydrolysing reagent and cholesterol oxidase or cholesterol dehydrogenase, as well as means for measuring the amount of cholesterol which reacts with the oxidase or dehydrogenase.

In a preferred embodiment, the kit comprises a device for the electrochemical determination of the HDL-cholesterol content. In this embodiment, the means for determining the amount of cholesterol which has reacted includes an electrochemical cell having a working electrode, a reference electrode or pseudo reference electrode and optionally a separate counter electrode; a power supply for supplying a potential across the cell; and a measuring instrument for measuring the resulting electrochemical response, typically the current across the cell.

The device for electrochemical determination also typically includes a reagent mixture as described above. The reagents may be present in the kit individually or in the form of one or more reagent mixtures. A single regent mixture is preferred. The reagent mixture may be present in the device in either liquid or solid form, but is preferably in solid form.

Typically, the reagent mixture is inserted into or placed onto the device whilst suspended/dissolved in a suitable liquid (e.g. water or buffer) and then dried in position. This step of drying the material into/onto the device helps to keep the material in the desired position. Drying may be carried out, for example, by air-drying, vacuum drying, freeze drying or oven drying (heating), preferably by freeze drying. The reagent mixture is typically located in the vicinity of the electrodes, such that when the sample contacts the reagent mixture, contact with the electrodes also occurs.

In one embodiment of the invention, the electrochemical cell is in the form of a receptacle. The receptacle may be in any shape as long as it is capable of containing a liquid which is placed into it. For example, the receptacle may be cylindrical.

Generally, a receptacle will contain a base and a wall or walls which surround the base. In this embodiment, the material comprising a transition metal salt is typically located in the receptacle.

Alternatively, the cell may be in the form of a partial receptacle. In this embodiment, the cell is designed such that when placed against a separate substrate, the partial receptacle together with the substrate forms a receptacle. In this embodiment, the partial receptacle comprises a wall or walls which connect a first open part with a second open part. The second open part may be placed against a substrate to form a receptacle, such that the substrate forms the true base of the receptacle thus formed. The second open part may, if desired, be covered by a permeable or semi-permeable membrane.

It is preferred that the electrochemical cell has at least one microelectrode. Typically, the working electrode is a microelectrode. For the purposes of this invention, a microelectrode is an electrode having at least one working dimension not exceeding 50 μm. The microelectrodes of the invention may have a dimension which is macro in size, i.e. which is greater than 50 μm.

The ‘working dimension’ of the electrode is one which is in contact with the test solution during operation. Further, the working dimension is one which causes the electrode to have an electrochemical response which at least in part corresponds to the typical response of a true microelectrode. Without wishing to be bound by any particular theory, an electrode can be considered to have an electrochemical response which is the sum of its ‘micro’ characteristic response (radial diffusion to the electrode) and its ‘macro’ characteristic response (semi-infinite diffusion to the electrode). In context of this invention, when determining the electrochemical response 5 seconds after application of a potential using a solution having a 4 cp viscosity, a ‘microelectrode’ will typically have a response of which at least 50%, preferably at least 60%, more preferably at least 70%, is determined by the ‘micro’ behaviour of the electrode.

An electrochemical cell may be either a two-electrode or a three-electrode system. A two-electrode system comprises a working electrode and a pseudo reference electrode. A three-electrode system comprises a working electrode, a reference electrode and a separate counter electrode. As used herein, a reference or pseudo reference electrode is an electrode that is capable of providing a reference potential. A pseudo reference electrode also acts as the counter electrode and is able to pass a current without substantially perturbing the reference potential.

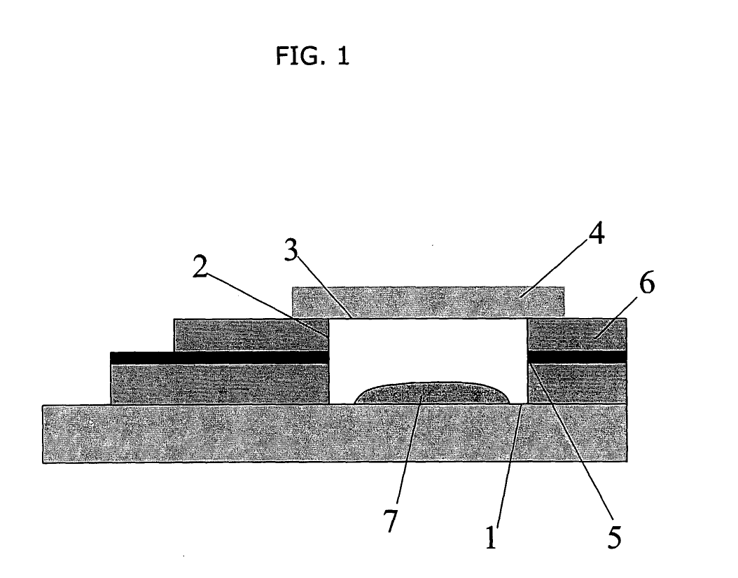

A device according to one embodiment of the invention is depicted in FIG. 1. In this embodiment, the working electrode 5 is a microelectrode. The cell is in the form of a receptacle or a container having a base 1 and a wall or walls 2. Typically, the receptacle will have a depth (i.e. from top to base) of from 25 to 1000 μm. In one embodiment, the depth of the receptacle is from 50 to 500 m, for example from 100 to 250 μm. In an alternative embodiment, the depth of the receptacle is from 50 to 1000 μm, preferably from 200 to 800 μm, for example from 300 to 600μm. The length and width (i.e. from wall to wall), or in the case of a cylindrical receptacle the diameter, of the receptacle is typically from 0.1 to 5 mm, for example 0.5 to 1.5 mm, such as 1 mm.

The open end of the receptacle 3 may be partially covered by an impermeable material or covered by a semi-permeable or permeable material, such as a semi-permeable or permeable membrane. Preferably, the open end of the receptacle is substantially covered with a semi-permeable or permeable membrane 4. The membrane 4 serves, inter alia, to prevent dust or other contaminants from entering the receptacle.

The membrane 4 is made of a material through which the sample to be tested can pass. For example, if the sample is plasma, the membrane should be permeable to plasma. The membrane also preferably has a low protein binding capacity. Suitable materials for use as the membrane include polyester, cellulose nitrate, polycarbonate, polysulfone, microporous polyethersulfone films, PET, cotton and nylon woven fabrics, coated glass fibres and polyacrylonitrile fabrics. These fabrics may optionally undergo a hydrophilic or hydrophobic treatment prior to use. Other surface characteristics of the membrane may also be altered if desired. For example, treatments to modify the membrane's contact angle in water may be used in order to facilitate flow of the desired sample through the membrane. The membrane may comprise one, two or more layers of material, each of which may be the same or different. For example, conventional double layer membranes comprising two layers of different membrane materials may be used.

The membrane may also be used to filter out some components which are not desired to enter the cell. For example, some blood products such as red blood cells, erythrocytes and/or lymphocytes may be separated out in this manner such that these particles do not enter the cell. Suitable filtration membranes, including blood filtration membranes, are known in the art. Examples of blood filtration membranes are Presence 200 and PALL BTS SP300 of Pall filtration, Whatman VF2, Whatman Cyclopore, Spectral NX and Spectral X. Fibreglass filters, for example Whatman VF2, can separate plasma from whole blood and are suitable for use where a whole blood specimen is supplied to the device and the sample to be tested is plasma.

For the purposes of this embodiment of the invention, the sample is the material which (when mixed with the reagent mixture) contacts the working electrode. In one embodiment, a specimen comprising the sample is supplied to the device of the invention and the specimen is filtered through the membrane prior to contacting the working electrode. For example, the specimen may be whole blood and the method may comprise the step of removing red blood cells from the specimen (e.g. using a blood filtration membrane) such that, for example, only plasma or serum contacts the working electrode. In this case, the sample is plasma or serum.

The electrochemical cell of this embodiment of the invention contains a working electrode 5 which is situated in a wall of the receptacle. The working electrode is, for example, in the form of a continuous band around the wall(s) of the receptacle. The thickness of the working electrode is typically from 0.01 to 25 μm, preferably from 0.05 to 15 μm, for example 0.1 to 20 μm. Thicker working electrodes are also envisaged, for example electrodes having a thickness of from 0.1 to 50 μm, preferably from 5 to 20 μm. The thickness of the working electrode is its dimension in a vertical direction when the receptacle is placed on its base. The thickness of the working electrode is its effective working dimension, i.e. it is a dimension of the electrode which contacts the sample to be tested. The working electrode is preferably formed from carbon, palladium, gold or platinum, for example in the form of a conductive ink. The conductive ink may be a modified ink containing additional materials, for example platinum and/or graphite. Two or more layers may be used to form the working electrode, the layers being formed of the same or different materials.

The cell also contains a pseudo reference electrode which may be present, for example, in the base of the receptacle, in a wall or walls of the receptacle or in an area of the device surrounding or close to the receptacle. The pseudo reference electrode is typically made from Ag/AgCl, although other materials may also be used. Suitable materials for use as the pseudo reference electrode will be known to the skilled person in the art. In this embodiment, the cell is a two-electrode system in which the pseudo reference electrode acts as both counter and reference electrodes. Alternative embodiments in which the cell comprises a reference electrode and a separate counter electrode can also be envisaged.

The pseudo reference (or reference) electrode typically has a surface area which is of a similar size to or smaller than, or which is larger than, for example substantially larger than, that of the working electrode 5. Typically, the ratio of the surface area of the pseudo reference (or reference) electrode to that of the working electrode is at least 1:1, for example at least 2:1 or at least 3:1. A preferred ratio is at least 4:1. The pseudo reference (or reference) electrode may, for example, be a macroelectrode. Preferred pseudo reference (or reference) electrodes have a dimension of 0.01 mm or greater, for example 0.1 mm or greater. This may be, for example, a diameter of 0.1 mm or greater. Typical areas of the pseudo reference (or reference) electrode are from 0.001 mm² to 100 mm², preferably from 0.1 mm² to 60 mm², for example from 1 mm² to 50 mm². The minimum distance between the working electrode and the pseudo reference (or reference) electrode is, for example from 10 to 1000 μm.

In order that the cell can operate, the electrodes must each be separated by an insulating material 6. The insulating material is typically a polymer, for example, an acrylate, polyurethane, PET, polyolefin, polyester or any other stable insulating material. Polycarbonate and other plastics and ceramics are also suitable insulating materials. The insulating layer may be formed by solvent evaporation from a polymer solution. Liquids which harden after application may also be used, for example varnishes. Alternatively, cross-linkable polymer solutions may be used which are, for example, cross-linked by exposure to heat or UV or by mixing together the active parts of a two-component cross-linkable system. Dielectric inks may also be used to form insulating layers where appropriate. In an alternative embodiment, an insulating layer is laminated, for example thermally laminated, to the device.

The electrodes of the electrochemical cell may be connected to any required measuring instruments by any suitable means. Typically, the electrodes will be connected to electrically conducting tracks which are, or can be, themselves connected to the required measuring instruments.

The required reagents are typically contained within the receptacle, as depicted at 7 in FIG. 1. Typically, the reagents, in the form of a single reagent mixture, are inserted into the receptacle in liquid form and subsequently dried to help immobilise the composition. The reagent mixture, for example, may be air-dried, vacuum dried, freeze dried or oven-dried (heated), most preferably it is freeze dried. On introduction of the sample into the receptacle, the dried material is re-suspended forming a liquid comprising the reagents and the sample, the liquid being in contact with the working electrode which is located in the wall of the receptacle. The liquid is also typically in contact with the reference and counter electrodes (3-electrode system) or with the pseudo reference electrode (2-electrode system). Thus, on application of a voltage across the cell, electrochemical reaction may occur and a measurable current be produced. Typically, a wet-up time, for example of 20 seconds, or from 1 second to 5 minutes, where a membrane is present over the receptacle, is provided before a voltage is applied, to allow the dried material to re-suspend.

The receptacle may, for example, contain one or more small air-holes in its base or its wall or walls (not depicted in FIG. 1). These holes allow air to escape from the receptacle when sample enters the receptacle. If such air-holes are not present, the sample may not enter the receptacle when it flows over the open end, or it may enter the receptacle only with difficulty. The air holes typically have capillary dimensions, for example, they may have an approximate diameter of 1-600 μm, for example from 100 to 500 μm. The air holes should be sufficiently small that the sample is substantially prevented from leaving the receptacle through the air holes due to surface tension. Typically, from 1 to 4 air holes may be present.

The cell may optionally comprise a separate counter electrode in addition to the working and reference electrodes. Suitable materials for producing the counter electrode will be known to the skilled person in the art. Ag/AgCl is an example of a suitable material.

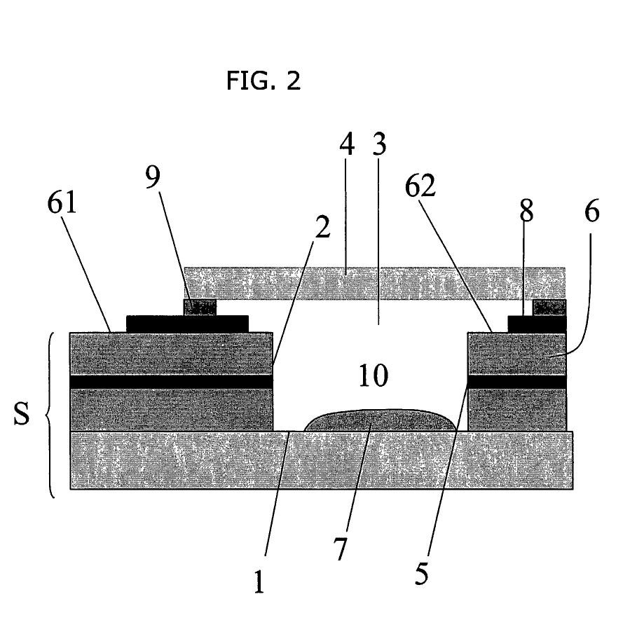

An alternative device for electrochemical determination of HDL-cholesterol is depicted in FIG. 2. The device of this embodiment is the same as the device depicted in FIG. 1 and described above, except as set out below. In this embodiment, the device comprises a strip S. The strip S may have any shape and size, but typically has a first surface 61, 62 which is substantially flat. The strip comprises a receptacle 10 bounded by base 1 and wall or walls 2. The device further comprises an electrochemical cell having a working electrode 5 in the wall(s) of the receptacle. The working electrode is typically a microelectrode. The device of this embodiment comprises a pseudo reference electrode acting as reference electrode and also as counter electrode. Alternatively, separate counter and reference electrodes may be used. The pseudo reference (or reference) electrode comprises a pseudo reference (or reference) electrode layer 8 present on the first surface of the strip 61, 62. The first surface of the strip is an external surface, i.e. it is a surface exposed to the outside of the device rather than a surface exposed to the interior of the receptacle. Typically, the pseudo reference (or reference) electrode layer substantially surrounds the receptacle or partial receptacle 10. As depicted in FIG. 2, it is preferred that the pseudo reference (or reference) electrode layer is not in contact with the perimeter of the first open part 3. Typically, the pseudo reference (or reference) electrode layer is at a distance of at least 0.1 mm, preferably at least 0.2 mm from the perimeter of the first open part. At least a part of the pseudo reference (or reference) electrode is, however, typically no more than 2 mm, for example no more than 1 mm or 0.5 mm, preferably no more than 0.4 mm from the perimeter of the first open part. In one embodiment, the pseudo reference (or reference) electrode substantially surrounds the receptacle or partial receptacle at a distance of from 0.01 to 1.0 mm, for example from 0.1 to 0.5 mm, or 0.2 to 0.4 mm from the perimeter of the first open part. Alternatively, this distance may be from 0.01 to 0.3 mm or from 0.4 to 0.7 mm.

The thickness of the pseudo reference (or reference) electrode is typically similar to or greater than the thickness of the working electrode. Suitable minimum thicknesses are 0.1 μm, for example 0.5, 1, 5 or 10 μm. Suitable maximum thicknesses are 50 μm, for example 20 or 15 μm.

The pseudo reference (or reference) electrode 8 typically has a surface area which is of a similar size to (or smaller than), or which is larger than, for example substantially larger than, that of the working electrode 5. Typically, the ratio of the surface area of the pseudo reference (or reference) electrode to that of the working electrode is at least 1:1, for example at least 2:1 or at least 3:1 preferably at least 4:1. The pseudo reference (or reference) electrode may, for example, be a macroelectrode. Where the ratio of the surface area of the pseudo reference (or reference) electrode to that of the working electrode is greater than 1:1, this helps to ensure that the electrochemical reaction occurring at the pseudo reference (or reference) electrode is not current-limiting. The actual area of the pseudo reference (or reference) electrode is, for example, from 0.001 mm² to 100 mm² or from 0.01 mm² to 60 mm², e.g. from 0.1 mm² to 60 mm², for example up to 50 mm² or 10 mm².

A membrane 4 may be attached to the device by any suitable attachment means 9, for example using a double-sided adhesive tape. Typically, the attachment means attaches the membrane to the first surface of the strip or to the pseudo reference (or reference) electrode layer. In a preferred embodiment as depicted in FIG. 2, the membrane is attached to the pseudo reference (or reference) electrode layer 8 at a location which is remote from the perimeter of the receptacle itself. Further, the attachment means is at a greater distance from the first open part of the receptacle 3 than the pseudo reference (or reference) electrode layer, such that at least a part of the surface of the pseudo reference (or reference) electrode layer close to or surrounding the receptacle is exposed to a sample which has passed through the membrane. Preferably, the attachment means is at least 0.2 mm, for example at least 0.3 mm or at least 0.4 mm, from the perimeter of the receptacle.

In the embodiment depicted in FIG. 2, a reaction volume is defined by the receptacle base 1 and walls 2, part of the surface of the strip 61, 62, the pseudo reference (or reference) electrode layer 8, the attachment means 9 and the membrane 4. This reaction volume can be varied by changing the volume of the receptacle, the position and thickness of the pseudo reference (or reference) electrode layer and the position and thickness of the attachment means 9. Preferred reaction volumes are at least 0.05 μl, for example at least 0.1 μl or 0.2 μl. It is further preferred that the reaction volume is no more than 25 μl, preferably no more than 5 μl, for example no more than 2 μl or no more than 1 μl. A typical reaction volume is approximately 0.8 μl.

The devices depicted in FIGS. 1 and 2 comprise receptacles having a base 1. In an alternative embodiment of the invention the base 1 may be absent such that a second open part is located at 1. In this embodiment, the device comprises a partial receptacle. Optionally, a permeable or semi-permeable membrane is placed over the second open part, for example a hydrophobic breathable membrane, e.g. Pall Versapor by Pall Filtration.

Further details regarding electrochemical cells which can be used in the devices of the present invention can be found in International Application No. PCT/GB05/002587 (and its priority claiming application GB application number 0414546.2). The contents of this application are incorporated herein by reference in its entirety.

The devices of the invention may comprise two or more electrochemical cells. Each cell comprises a working electrode and may additionally comprise a counter electrode. Alternatively, two or more adjacent cells may employ the same counter electrode. This embodiment of the invention allows a number of measurements to be taken simultaneously. The multiple cells may all contain the same reagent mixture, in which case the separate measurements may be used to assist in the elimination of errors. Alternatively, different reagent mixtures may be located in each cell in order to provide measurements of a plurality of parameters. A control cell may also be provided. The kit of the invention may comprise a strip S containing the electrochemical cell(s) (e.g. that depicted in FIG. 2 and described above) and an electronics unit, e.g. a hand-held portable electronics unit, capable of forming electronic contact with the strip S. The electronics unit may, for example, house the power supply for providing a potential to the electrodes, as well as a measuring instrument for detecting a current and any other measuring instruments required. One or more of these systems may be operated by a computer program.

The devices of the invention can be produced by forming a laminate structure comprising a layer of working electrode material (e.g. a layer of graphite) between two layers of insulating material. A hole is then punched (or drilled or cut) through this laminate, thus forming the wall(s) of the receptacle. A base, optionally comprising a counter electrode, is then added. The counter electrode may alternatively be provided by printing a layer of a suitable material onto the insulating material surrounding, or close to, the open part of the receptacle. Where an air hole is desired in the base or wall(s) of the receptacle, this can be formed by any suitable technique, for example by drilling or punching a hole, or by use of an air permeable membrane as the base. Full details regarding the process for producing cells as depicted in FIGS. 1 and 2 can be obtained from International Application No. PCT/GB05/002587 (and GB application number 0414546.2), which is referenced above.

The device of the present invention is operated by providing a sample to the device and enabling the sample to contact the reagent mixture. Typically a wet-up time of approximately 20 seconds is provided to enable the reagent mixture to be dissolved/suspended in the sample and to allow reaction to occur. The sample/reagent mixture should be in electronic contact with the working electrode in order that electrochemical reaction can occur at the electrode.

A potential is then applied across the cell and, typically, the current produced is measured. Typically, the potential is applied after a period of up to 3 minutes, e.g. up to 2 minutes, after providing the sample to the device. This period is preferably from 10 seconds to 180 seconds, e.g. from 10 seconds to 90 seconds, for example from 15 seconds to 1 minute, from 15 seconds to 30 seconds or approximately 20 seconds. The use of periods within this preferred range helps to ensure that the measurement detects only cholesterol bound to HDL. Where longer periods are used, some cholesterol bound to non-HDL lipoproteins, e.g. LDL, may also react leading to an inaccurate measurement of the HDL-cholesterol content.

Typically, where Ru(II) is the product to be detected at the working electrode, the potential applied to the cell is either from 0.1V to 0.3V, or −0.4V or lower, for example from −0.4V to −0.6V. Preferred applied potentials are 0.15V and −0.45V. (All voltages mentioned herein are quoted against a Ag/AgCl reference electrode). In a preferred embodiment, the potential is stepped first to a positive applied potential, e.g. 0.1 to 0.2V, for a period of about 1 second, and then stepped to a negative applied potential of −0.4 to −0.6V for a further 1 second. The use of the double potential step enables correction for electrode fouling and variation in electrode area to be minimized, as is described in WO 03/097860 (incorporated herein by reference in its entirety). Where a different redox agent is used, the applied potentials can be varied in accordance with the potentials at which the oxidation/reduction peak occurs.

### EXAMPLES

### Example 1

A device of the type depicted in FIG. 2, wherein the base 1 is formed by a membrane (Pall Versapor) was used. The working electrode is a carbon electrode and the pseudo reference electrode is a Ag/AgCl electrode. The volume defined by the walls, base, adhesive and bottom surface of the membrane 4 is approximately 0.8 μl. A reagent mixture is inserted into the partial receptacle of the device and freeze dried, prior to attachment of a Whatman VF2 membrane over the device at 4.

The reagent mixture comprises:
Ruthenium hexamine (Mwt 309.61). Sigma (Ru) (100 mM)
Nicotinamide adenine dinucleotide (NAD) (Mwt 663.4) Sigma (8 mM)
Putidaredoxin reductase (PDR) (2 mg/100 μl)
Cholesterol dehydrogenase -Toyobo (CHD) (6 mg/100 μl)
Purified Lipoprotein Lipase (PEG-modified) ((Psuedomonas) (PEG-Lipase) (12 mg/100 μl)
Emulgen B-66—KAO Chemicals (5%)
Tris buffer (0.1M) with mannitol/MgCl₂ (600 mM)/NaOH (8 mM)
Myo-Inositol, minimum 99%—Sigma MW 180.16 (1%)
Glycine (0.1%)
Ectoine (0.1%)
PEG-ylated BSA (5%)

A number of specimens having unknown HDL-cholesterol contents are supplied to the device in a series of experiments. For each Example, a wet-up period of 20 seconds is allowed to elapse to permit up-take of the reagents in the sample and reaction between the reagents and the sample. A potential of +0.15V is then applied for 1 second followed by a potential of −0.45V for a further 1 second. The current is measured and the amount of cholesterol bound to HDL in the sample calculated.

### Example 2

A device of the type described in Example 1 was used, except having a volume of 0.2 μl. The reagent mixture used was as follows:
Ruthenium hexamine (Mwt 309.61). Sigma (Ru)
Nicotinamide adenine dinucleotide (NAD) (Mwt 663.4) Sigma
Putidaredoxin reductase (PDR)
Cholesterol dehydrogenase—Toyobo (CHD)
Purified Lipoprotein Lipase (PEG-modified) ((Psuedomonas) (PEG-Lipase)
1M Tris-HCl pH8—Sigma
0.1M Tris pH9 buffer
Emulgen B-66—KAO Chemicals
Tris buffer with mannitol/MgCl₂
Myo-Inositol, minimum 99%—Sigma MW 180.16
D-Mannitol, SigmaUltra—Sigma MW 182.17
MgCl₂—Sigma Mw 203.3
Phosphotungstic acid—Sigma (PTA)
   providing a solution having the following concentrations:

| | |
|---|---|
| Ru = 100 mM | TRIS pH 8 (0.1M) |
| NAD = 8 mM | 10% Manitol |
| PDR = 1 mg/100 μl | MgCl₂ 200 mM |
| PEG-Lipase = 2 mg/100 μl | 20% B66 |
| CHD = 2 mg/100 μl | PTA = 0.4% wt/vol |

A number of specimens having unknown HDL-cholesterol contents were supplied to the device in a series of experiments (Examples 2a to 2i). For each Example, a wet-up period of one minute was allowed to elapse to permit up-take of the reagents in the sample and reaction between the reagents and the sample. A potential of +0.15V was then applied for 1 second followed by a potential of −0.45V for a further 1 second. The current was measured and the amount of cholesterol bound to HDL in the sample calculated. The results are shown in Table 1 below. Also shown in Table 1 are the results of a non-electrochemical test which is commercially available (Randox HDL Direct, Randox Laboratories, Ltd). This Table demonstrates that the electrochemical results correlate well with results achieved using known techniques.

TABLE 1

| | Method of the | Randox HDL | |
|---|---|---|---|
| | Invention | Test | |
| Example | (mM) | (mM) | Sample |
| 2a | 1.48 | 1.55 | Plasma |
| 2b | 1.33 | 1.37 | Plasma |
| 2c | 1.12 | 1.06 | Plasma |
| 2d | 1.01 | 0.91 | Plasma |
| 2e | 1.24 | 1.1 | Plasma |
| 2f | 1.1 | 0.93 | Plasma |
| 2g | 1.28 | 1.29 | Plasma |
| 2h | 1.30 | 2.24 | Plasma |
| 2i | 1.96 | 2.08 | Plasma |

The invention has been described with reference to various specific embodiments and examples. However, it is to be understood that the invention is in no way limited to these specific embodiments and examples.

1. A method for the determination of the amount of cholesterol in high density lipoproteins in a high density lipoprotein containing sample, said method comprising reacting the sample with (a) a PEG-ylated protein and subsequently measuring the amount of cholesterol in the high density lipoproteins.

2. A method according to claim 1, wherein the amount of cholesterol in the high density lipoproteins is measured by reacting the sample with (b) a cholesterol ester hydrolysing reagent and (c) cholesterol oxidase or cholesterol dehydrogenase and determining the amount of cholesterol which has reacted with the cholesterol oxidase or cholesterol dehydrogenase.

3. A method according to claim 1, wherein the sample is additionally reacted with (f) a surfactant.

4. A method according to claim 1, wherein the PEG-ylated protein is a PEG-ylated polyaminoacid, gelatine, lysozyme, lactalbumin or serum albumin.

5. A method according to claim 4, wherein the PEG-ylated protein is PEG-ylated bovine serum albumin.

6. A method according to claim 1, wherein the amount of cholesterol in the high density lipoproteins is measured by an electrochemical technique.

7. A method according to claim 6, wherein the method comprises reacting the sample with
(a) a PEG-ylated protein;
(b) a cholesterol ester hydrolysing reagent;
(c) cholesterol oxidase or cholesterol dehydrogenase;
(d) a coenzyme;
(g) a redox agent capable of being oxidised or reduced to form a product; and optionally
(h) a surfactant,
and electrochemically detecting the amount of product formed.

8. An electrochemical method for the determination of the amount of cholesterol in high density lipoproteins in a high density lipoprotein containing sample, said method comprising reacting the sample with
(b) a cholesterol ester hydrolysing reagent;
(c) cholesterol oxidase or cholesterol dehydrogenase;
(d) a coenzyme;
(e) a redox agent capable of being oxidised or reduced to form a product; and optionally
(f) a surfactant,
(h) a complexing reagent capable of forming a complex with low density and very low density lipoproteins; and/or
(i) a PEG-ylated protein;
and electrochemically detecting the amount of product formed, wherein the cholesterol ester hydrolysing reagent and/or cholesterol oxidase or cholesterol dehydrogenase is modified with polyethylene glycol.

9. A method according to claim 8, wherein the complexing agent (h) comprises a polyanion selected from phosphotungstic acid and salts thereof, dextran sulphuric acid and salts thereof, polyethylene glycol and heparin and salts thereof and optionally also comprises a divalent metal salt.

10. A method according to claim 8, wherein the complexing reagent (h) comprises an antibody capable of binding to apoB-containing lipoproteins.

11. A method according to claim 8, one of claims 8 to 10, wherein the cholesterol ester hydrolysing reagent (b) is an optionally PEG-ylated cholesterol esterase or lipase.

12. A method according to claim 11 wherein component (b) is an optionally PEG-ylated cholesterol esterase or lipase and component (c) is optionally PEG-ylated cholesterol dehydrogenase.

13. A method according to claim 8, wherein the sample is additionally reacted with (g) a reductase.

14. (canceled)

15. A method according to claim 8, wherein the coenzyme (d) is NAD⁺ or an analogue thereof.

16. (canceled)

17. A method according to claim 8, wherein the sample is reacted simultaneously with the cholesterol ester hydrolysing reagent, cholesterol oxidase or cholesterol dehydrogenase and, if used, the PEG-ylated protein or complexing reagent.

18. (canceled)

19. A reagent mixture for use in a method for the determination of the amount of cholesterol in high density lipoproteins in a high density lipoprotein containing sample, the reagent mixture comprising
(a) a PEG-ylated protein;
(b) a cholesterol ester hydrolysing reagent;
(c) cholesterol oxidase or cholesterol dehydrogenase; and optionally
(f) a surfactant.

20. A reagent mixture according to claim 19, which additionally comprises (d) a coenzyme, (e) a redox agent capable of being oxidised or reduced to form a product; and optionally (g) a reductase.

21. A reagent mixture for use in an electrochemical method for the determination of the amount of cholesterol in high density lipoproteins in a high density lipoprotein containing sample, the reagent mixture comprising
(b) a cholesterol ester hydrolysing reagent;
(c) cholesterol oxidase or cholesterol dehydrogenase;
(d) a coenzyme;
(e) a redox agent capable of being oxidised or reduced to form a product; and optionally
(h) a complexing reagent capable of forming a complex with low density and very low density lipoproteins; and/or
(i) a PEG-ylated protein;
wherein the cholesterol ester hydrolysing reagent and/or the cholesterol oxidase or cholesterol dehydrogenase is modified with polyethylene glycol.

22. A reagent mixture according to claim 21, which additionally comprises (f) a surfactant and/or (g) a reductase.

23. A kit for the determination of the amount of cholesterol in high density lipoproteins in a high density lipoprotein containing sample, the kit comprising (a) a PEG-ylated protein capable of selectively complexing with non-HDL lipoproteins, (b) a cholesterol ester hydrolysing reagent, (c) cholesterol oxidase or cholesterol dehydrogenase, optionally (f) a surfactant, and means for measuring the amount of cholesterol which reacts with the cholesterol oxidase or cholesterol dehydrogenase.

24. A kit for the determination of the amount of cholesterol in high density lipoproteins in a high density lipoprotein containing sample, the kit comprising
an electrochemical cell having a working electrode, a reference or pseudo reference electrode and optionally a separate counter electrode;
the reagents defined in claim;
a power supply for applying a potential across the cell; and
a measuring instrument for measuring the resulting electrochemical response.

25. A kit according to claim 24, wherein the reagents are present as a single reagent mixture.

26. A kit according to claim 25, wherein the reagent mixture is in dried form.

27. A kit according to claim 24, wherein the working electrode is a microelectrode having at least one dimension of less than 50 μm.

28. A kit according to claim 24, wherein the electrochemical cell is in the form of a receptacle or partial receptacle, the working electrode is in a wall of the receptacle or partial receptacle and the reagents are at least partly contained within the receptacle or partial receptacle.

29. A kit according to claim 24 comprising a strip having at least one receptacle or partial receptacle formed therein, the receptacle or partial receptacle having a first open part in a first surface of the strip to enable a sample to enter the receptacle or partial receptacle,
wherein the working electrode of the electrochemical cell is in a wall of the receptacle or partial receptacle, and
wherein the reference or pseudo reference electrode of the electrochemical cell comprises a reference or pseudo reference electrode layer formed on at least a part of the first surface of the strip, and
wherein the reagents are at least partly contained within the receptacle or partial receptacle.

30. A kit for the determination of the amount of cholesterol in high density lipoproteins in a high density lipoprotein containing sample, the kit comprising
an electrochemical cell having a working electrode, a reference or pseudo reference electrode and optionally a separate counter electrode;
the reagents defined in claim 21;
a power supply for applying a potential across the cell; and
a measuring instrument for measuring the resulting electrochemical response.

31. A method of operating a kit as defined in claim 24, said method comprising
(i) contacting (1) the reagents and (2) a high density lipoprotein containing sample, with each other and with the electrodes;
(ii) applying a potential across the electrochemical cell; and
(iii) electrochemically detecting the amount of product formed by measuring the resulting electrochemical response.

32. (canceled)
